(19) United States
(12) Patent Application Publication
(10) Pub. No.: US 2021/0100667 A1
(43) Pub. Date: Apr. 8, 2021

(54) Braided Stent Processes & Methodologies for Making and Using the Same for Improved Access & Outcomes

(71) Applicant: TREADSTONE HOLDINGS, LLC, Lake Forest, CA (US)

(72) Inventors: Tuan Ngo, Anaheim, CA (US); Randall Takahashi, Mission Viejo, CA (US)

(21) Appl. No.: 16/988,698

(22) Filed: Aug. 9, 2020

Related U.S. Application Data
(60) Provisional application No. 62/884,623, filed on Aug. 8, 2019.

(51) Int. Cl. A61F 2/89; A61F 2/90

(52) U.S. Cl. CPC A61F 2/89; A61F 2/90; A61F 2220/0016; A61F 2250/0098; A61F 2220/005; A61F 2210/0014; A61F 2220/0058

(57) ABSTRACT

One wire woven stent, created around pins and grooved channels and laser welded at the ends, according to the patterns whereby said pattern can be started at any pins and continued, with improved performance and access to tortuous vessels.

### CROSS REFERENCE TO RELATED APPLICATIONS

The application claims full priority benefit from U.S.Ser. No. 62/884,623, filed Aug. 8, 2019. Likewise, expressly incorporated by reference are the following: U.S. Pat. Nos. 9,198,687; 8,945,172; 8,574,262; 10,722,242; 10,342,546; 10,729,447; and 9,566,072, the content of which is incorporated herein by reference herein in its entirety.

### FIELD

The present disclosures relate to the minimally invasive delivery of stents in the neurovascular region and throughout the body.

Although the LVIS® and LEO® stents have been developed, needs for improved braided stents remain patent and available, particularly using one-wire woven and laser welded-wise, which would overcome both navigability and foreshortening issues—while providing better hand for those emplacing them. Both of the prior stents (LVIS® and LEO®) and all of their respective IP, marketing and literature depictions are expressly incorporated herein by reference, and all of the public domain.

### BACKGROUND OF THE DISCLOSURES

The present inventors, have identified viable weave patterns and prototyped an improved braided stent.

### BRIEF DESCRIPTION OF THE DRAWINGS

The patent or application file contains at least one drawing executed in color.

Various preferred embodiments are described herein with references to the drawings in which merely illustrative views are offered for consideration, whereby:

FIG. 1 is a braided stent according to the invention being woven;

FIG. 2 is the same; and

FIG. 3 is a finished braided stent.

Corresponding reference characters indicate corresponding components throughout the several views of the drawings. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### DETAILED DESCRIPTIONS

The present inventors have re-tooled both the apparatus and approach to making braided stents. Offered herein is a novel way to make a more flexible, yet compliant member to treat neurovascular challenges. By combining multi-crown sides with easily replicable weave patterns, a stent is created to navigate tight spaces, while non-foreshortening enough to accommodate ⅔rd's of its length being released and withdrawn.

Forced Expansion:

Using an under over method at the end crowns, the end crowns work together to ensure optimal expansion possible to pull back the braided stent into the catheter with only ˜10% remaining where the competition can only allow ˜30% remaining allowing for more “last minute” positioning over the competition.

Tooling:

The fixture allows for easy finding of Pin number;

Collets can be moved to accommodate an infinite amount of stent lengths; and

Collets and weave rod can be sized to make an infinite amount of stent diameters.

A single nitinol wire or single DFT wire will be used to make the entire braided stent. The wire will be braided in a specific weave pattern utilizing numbered pins (ex. 16 pins) to completion at the same location where the braid began. The wire will be laser welded together at that location. A marker band can be conveniently placed over this location in order to disguise the laser weld.

Marker Bands: The braided stent will utilize the usage of marker bands/coils to ensure radiopacity of the braid. The marker bands will be set in place using adhesive/welding. Secondly, a single or multiple tantalum wire will allow for secondary radiopacity. Stent Pattern with Tantalum Wire.xlsx gives an example of where the marker bands will be placed.

Different Sizes: Almost any size stent in length or diameter can be possible. The weave pattern is flexible to be repeated over the entire length. The number of revolutions (targeting 2-3 revolutions) can be expanded to ensure different tightness of braid. The wire diameter can be interchanged for larger or smaller stents leaving the possibilities of size and length infinite combinations.

Weave Pattern:

See Stent Pattern with Tantalum Wire.

Different Weave Pattern which aids in maximum opening efficiency along with the end crowns.

Weave pattern is easier to remember for the operators.

Can return back to the pattern from a break and restart with ease.

Pattern can be started at any pins and continued (e.g. Start at #2 but follow #1 instructions, etc.).

.xlsx for weave pattern (U=Under, O=Over).

Forced Expansion:

Using an under over method at the end crowns, the end crowns work together to ensure optimal expansion possible to pull back the braided stent into the catheter with only ˜10% remaining where the competition can only allow ˜30% remaining allowing for more “last minute” positioning over the competition.

Tooling:

The fixture allows for easy finding of Pin number.

Collets can be moved to accommodate an infinite amount of stent lengths.

Collets and weave rod can be sized to make an infinite amount of stent diameters.

Rod mounted on a moveable collet with a bearing.

Materials are able to handle high temperatures (Inconel, titanium, nitinol, steel alloys, DFT comprised of nitinol and platinum).

### SUMMARY OF THE INVENTION

According to embodiments there is disclosed a one wire woven stent, created around pins and grooved channels and laser welded at the ends.

Braided Stent Distinguishing Factors:

The braided stent consists of the following materials: Wire, Marker bands/Marking Coils, and a Radiopaque wire.

### EXAMPLE 1: Completed Braided Stent

The present inventors have improved upon implementation of proprietary weave patterns imparting better performance than existing LVIS®/LEO® stents. Unexpectedly, the weave patterns developed enable operators/weavers/users to more easily keep track of steps and progress, while imparting also unexpectedly improved performance.

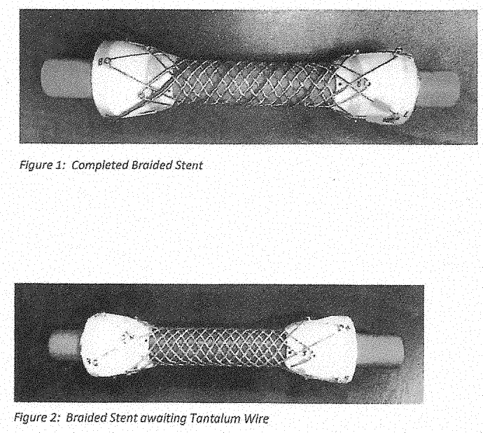

Referring now to FIG. 1 and FIG. 2, the braided stent is made of Nitinol wire or DFT wire, using for example 16 pins to completion at the same location where the braid began, in an embodiment the wires in laser welded at that location, a maker band is emplaced. The braided stent will utilize the usage of marker bands/coils to ensure radiopacity of the braid unless DFT is used. The marker bands will be set in place using adhesive. Secondly, a single or multiple tantalum wire will allow for secondary radiopacity. Stent Pattern with Tantalum Wire.xlsx gives an example of where the marker bands will be placed.

Different Sizes: Almost any size stent in length or diameter can be possible. The weave pattern is flexible to be repeated over the entire length. The number of revolutions (targeting 2-3 revolutions) can be expanded to ensure different tightness of braid. The wire diameter can be interchanged for larger or smaller stents leaving the possibilities of size and length infinite combinations.

As shown in FIG. 3, three differences are shown over prior art (eg. LVIS types of stents), i) access is enabled by the “slinky-like” malleability imparted by the weave pattern “enables the braided stent to slink about tortuous anatomy” (PI pre-clinical trials); ii) greater than ⅔s of any subject stent may be deployed, unsheathed and then re-withdrawn without performance decrease at all; and iii) DFT (drawn filled tube) malleability allows for the desired issue-free detachment each time the stent is deployed.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

FIG. 3 further illustrates that improved malleability and extra crowns-per-side allow users to better manipulate and oppose the instant system within a patient. For example, because the braided stent of the present inventions flexes instead of breaks or being weakened, even the most challenged vessels may be accessed and bridged, alone or in combination with coils.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language mans that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O mechanism may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antenna, and a network interface device (e.g., a network interface card (NIC), Wi-Fi card, cellular modem, data jack, Ethernet port, modem jack, HDMI port, mini-HDMI port, USB port), touchscreen (e.g., CRT, LCD, LED, AMOLED, Super AMOLED), pointing device, trackpad, light (e.g., LED), light/image projection device, or a combination thereof.

Memory according to the invention refers to a non-transitory memory which is provided by one or more tangible devices which preferably include one or more machine-readable medium on which is stored one or more sets of instructions (e.g., software) embodying any one or more of the methodologies or functions described herein. The software may also reside, completely or at least partially, within the main memory, processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the invention includes a tangible, non-transitory computer readable medium for memory. Exemplary devices for use as memory include semiconductor memory devices, (e.g., EPROM, EEPROM, solid state drive (SSD), and flash memory devices e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

One wire woven stent, created around pins and grooved channels and laser welded at the ends, disclosed or described herein and/or shown or an obvious permutation of the same or hybrid with existing technology for the neurovasculature.

What is claimed:

1. An improved device for the treatment of aneurysms, particularly those located at or distal to the circle of Willis and not amenable to coiling; which comprises a radiopacity showing single wire woven low-profile braided stent, mitigating risk of interprocedural failure based on hemodynamic parameters.

2. The improved device of claim 1, further comprising at least one of a single nitinol wire and a single DFT wire.

3. The improved device of claim 2, further comprising marker bands employed via at least one of adhesive and welding.

4. The improved device claim 3, subject tooling consisting essentially of at least material able to handle high temperature from the group of: Inconel, titanium, nitinol, steel alloys and DFT comprised of nitinol and platinum.

5. The improved device of claim 4, created around pins and grooved channels.

6. The improved device of claim 5, being laser welded at the ends.

7. The improved device of claim 6, further comprising, having a weave pattern which is easier to remember for operators, in that the operator may return back to the pattern and re-start after halting with low risk of error.

8. A method of creating a braided stent of specified size in length and/or diameter, which comprises, in combination:
braiding a single wire in a specific weave pattern utilizing pre-set number of pins.

9. The method of claim 8, where the terminal number, for example, 16 pins ends at the same location where the braid began.

10. The method of claim 9, the wire being laser welded together at the denominated location.

11. The method of claim 9, the wire being adhesed together at the denominated location.

12. The method of claim 10, wherein a marker band can be placed over the denominated location to cover the laser weld.

13. The method of claim 12, further comprising a flexible weaving pattern to be repeated over the entire length.

14. The method of claim 13, wherein 2-3 revolutions, can be expanded to ensure different tightness of braid.

15. The method of claim 14, wherein the wire diameter can be inter-changed for larger or smaller stents leaving the possibilities of size and length of nearly infinite combinations.

16. A novel woven tantalum wire stent, wherein the weave pattern is .x1sx.

17. The novel woven stent of claim 16, wherein the weave pattern results in maximum opening efficiency owing to at least 6-9 end crowns.

18. The novel woven stent of claim 17, wherein an operator can return back to pattern after a break & restart with ease, not losing their place.

19. The novel woven stent number claim 18, further comprising a tantalum wire and per side of 6 to 9 crowns.

20. An improved device of claim 7, wherein using an under over method of the end crowns, the end crowns work together to ensure optimal expansion possible to pullback the braided stent into catheter with only 10% remaining for last minute repositioning and adjustment.
